(19) United States
(12) Patent Application Publication
(10) Pub. No.: US 2018/0327857 A1
(43) Pub. Date: Nov. 15, 2018

(54) DIAGNOSTIC BIOMARKER AND DIAGNOSTIC METHOD

(71) Applicant: Shanghai Realgen Biotech Co., Ltd., Shanghai (CN)

(72) Inventors: Youping DENG, Honolulu, HI (US); Hongwei WANG, Shanghai (CN)

(21) Appl. No.: 15/924,907

(22) Filed: Mar. 19, 2018

(30) Foreign Application Priority Data
May 9, 2017 (CN) 201710321597.3
May 9, 2017 (CN) 201710322017.2

(51) Int. Cl. C12Q 1/6886; G01N 33/574; C12Q 1/6809

(52) U.S. Cl. CPC C12Q 1/6886; G01N 33/57423; C12Q 1/6809; C12Q 2600/106; C12Q 2600/158; C12Q 2600/118; G01N 2800/7028

(57) ABSTRACT

The present invention relates to a diagnostic biomarker, a method for identifying the diagnostic biomarker, and a diagnostic method using the diagnostic biomarker. Specifically, the present invention uses a ratio of ncRNAs as a diagnostic biomarker, identifies optimal ncRNAs pair associated with diseases based on SVM-RFE algorithm, and then uses the same for diagnosis of the diseases.

### FIELD OF THE INVENTION

The present invention belongs to a field of diagnosis or detection of a disease. Specifically, the present invention relates to a ratio-based biomarker, its identification method, and a method for diagnosing a disease using the biomarker. More specifically, the present invention relates to a method for identifying non-coding RNA (abbreviated as ncRNA or plural ncRNAs) pairs in plasma, and in particular ncRNA pairs capable of distinguishing healthy control samples from lung adenocarcinoma, and identification method.

### BACKGROUND OF THE INVENTION

Micro RNAs (miRNAs) are endogenous, small non-coding RNAs, usually 18-25 nucleotides long. They have been found to play crucial roles in post-transcriptional regulation of mRNA. MiRNAs play a pivotal role in cell differentiation, proliferation, and apoptosis and are implicated in many types of disease including cancer, diabetes, cardiovascular and neural diseases. Besides miRNAs, there are other small non-coding RNAs (ncRNAs) important in regulating gene expression at many levels, such as chromatin architecture, transcription, mRNA stability and translation, including small snoRNAs, Piwi-interacting RNAs (piRNAs), short interfering RNAs (siRNAs), and tRNAs shown to be perturbed in cancer and other diseases. For instance, snoRNAs comprise a highly abundant group of small ncRNAs, and a limited number of snoRNAs have been reported to have ncRNA-like functions in gene splicing and silencing. Recent studies have demonstrated that three snoRNAs displayed altered expression in non-small cell lung cancer (NSCLC) patients, and SNORA42 may act as an oncogene in lung tumorigenesis.

During recent years, a series of studies have shown that miRNAs can also be detected in body fluids such as serum, plasma, saliva, milk, sputum, and urine, and circulating miRNAs have been detected packaged in exosomes or microvesicles (MVs), or bound to specific proteins such as Ago-2. Once in the extracellular space, miRNAs could be taken up by other cells (cell-to-cell communication), degenerated by RNases, or excreted. Even though the mechanism of secretion and incorporation of miRNAs has not been fully understood, circulating miRNAs may be involved in physiological and pathological events.

These findings opened a door for circulating ncRNAs as non-invasive biomarkers for diagnostics and prognostics of different kinds of diseases. Due to high sensitivity, specificity and low template requirements, currently, the most currently used method for measuring circulating miRNAs is reverse transcription quantitative PCR (RT-qPCR). Because of very low concentration of circulating RNAs in the body fluids, accurately measuring circulating miRNA expression is a great challenge. Moreover, similar to gene expression analysis, systematic factors such as variations in the amount of starting material, sample collection, RNA isolation, reverse transcription, and PCR will affect the final results and induce bias and quantitation error. So currently, normalization reference control molecules are used to normalize circulating miRNA PCR data in order to fairly evaluate circulating miRNA expression. Current reference control molecules include external and internal endogenous controls. Many researchers choose to use spike-in synthetic RNA sequence (like C. elegans miR-39 and miR-54, or plant miRNAs) as extremal reference controls for normalization of circulating miRNAqPCR analysis. A variety of internal controls have been used. For instance, one of small-nucleolar RNAs (snoRNAs), such as RNU6B was initially utilized to normalize circulating miRNA data, but was later found to be deregulated according to particular diseases and tumor prognosis. Many studies considered a reference miRNA, like miR-16, that was shown to have variation in plasma samples of cancer patients. Due to lack of consensus normalization methods, data consistency and reproducibility across different studies are often not comparable. Therefore, it is urgent to find the best normalization method for the circulating miRNA data.

Data normalization in plasma/serum ncRNA experiments using RT-qPCR is a challenge. Taking miRNA as an example, because the yield of total RNA from small-volume plasma or serum samples (i.e., 100 or 200 μl) was below the limit of accurate quantification by spectrophotometry, bias in sample collection, storage and processing also affects the accuracy and reliability of the quantitative analysis of circulating miRNA. The inclusion of an external or endogenous reference control molecule is recommended to adjust technical variations in the RNA recovery procedure by the current experiments. Many researchers chose to spiked-in synthetic RNA sequence (like C. elegans miR-39 and miR-54, or plant miRNAs) into the sample for normalization of circulating miRNA qPCR analysis. In our study, we chose C. elegans Cel-miR-54 as an external control, and we found it was not a good control in both sequencing and RT-qPCR data. The reason is that these synthetic miRNAs added directly to plasma were rapidly degraded and less stable than endogenous miRNAs when added to plasma, because they are not protected from endogenous RNase activity. However, circulating miRNAs are relatively stable as they are protected from endogenous RNase activity, either because they are bound to proteins or contained within endosomes.

Some researchers have made efforts to seek the suitable endogenous control miRNAs (ECM); however, no such suitable ECMs have been established for blood miRNA quantification. For example, miR-16 is frequently used as a control, but elevated levels of miR-16 in serum correlate with bone metastasis in patients with breast cancer and it was reported that endogenous miR-16 was a poor normalizing factor. Since Chen X et al. reported that let-7d/g/i is a good endogenous control for normalizing circulating miRNA data, we tested let-7d/g/i as endogenous control in the experiment. We found that they were not stably expressed across our samples. Chen's samples were only derived from a Chinese population although lung cancer patients were included, which could be a reason why we did not get the similar results. The widely used endogenous control has-MiR-191 did not work out as a good control in our experiment either. We could endlessly test more endogenous controls such as U6, RNU44, RNU48, miR-16, miR-103, and miR-23a that have been commonly utilized nowadays. However, Chen's paper has already found that these controls performed even worse than let-7d/g/i. A well known ideal endogenous reference control should at least meet the criteria that they are stably expressed across all samples and experimental conditions. It is very hard to prove which candidate endogenous molecule meets the criteria.

Using ratio as biomarkers has been applied to some diseases. For instance, the AB42/AB40 ratio has been a promising biomarker for Alzheimer's disease (AD), and Apo B/Al ratio is a much better biochemical indicator for people with obesity. Using the miRNA ratios as a tool for miRNA RT-qPCR data has been also reported in cancer biomarker papers. However, there is no specific report to recommend ratio based normalization method as a good way to normalize circulating ncRNA sequencing and RT-qPCR data. At present, almost 99% of publications involved in circulating ncRNAs (miRNAs) are still using external or internal reference control molecules for normalizing circulating PCR data. Some research are still desperately searching for better reference controls for normalizing circulating miRNA data.

Lung cancer is a common disease with most heterogeneity and is the No. 1 killing disease among the male cancers. Further, this cancer is susceptible to metastasis of regional lymph nodes and remote organs. New cases of lung cancer every year in the world account for 17% of all the new cases of cancers, and number of death due to lung cancer accounts for 23% of all deaths. In China, lung cancer is a most common cancer detected and also the first reason of death in cancers. Among males of 60-74 years old, lung cancer is most one newly incurred and also highest in terms of number of death in cancers. Lung cancer may be divided into small cell lung cancer and non-small cell lung cancer (NSCLC), wherein NSCLC is a malignant tumor with poor prognosis and high risk, accounts for 85% of lung cancer cases. NSCLC has two common subtypes: adenocarcinoma (about 70%) and squamous cell lung cancer (SqCC, about 30%). Metastasis has occurred in about ⅔ of patients upon the diagnosis. Therefore, early diagnosis and early treatment are critical to patients of lung cancer, wherein early diagnosis may decrease the death rate by 10-50 folds. Low dose spiral CT (LDCT) is currently an important means for a non-invasive screening of early stage of lung cancer. However, it usually produces a false positive result. Therefore, early detection of lung cancer still needs a microinvasive method, e.g., a molecular biological marker in plasma.

Recent studies have indicated that circulating noncoding RNAs (ncRNAs) such as miRNAs are stable and can be used as biomarkers for the diagnosis and prognosis of human diseases. However, due to the very low concentration of circulating ncRNAs in blood, data normalization in plasma/serum ncRNA experiments using next-generation sequencing and quantitative real time RT-PCR is a challenge. The current normalization methods based on synthetic external spiked-in controls or published endogenous miRNA controls were not appropriate, because they are not stably expressed and failed to find significantly reliable differentially-expressed ncRNAs. Towards lung adenocarcinoma, there is no clinically effective microinvasive/noninvasive marker suitable for early diagnosis.

### SUMMARY OF THE INVENTION

To overcome defects in the prior art, the present invention provides a novel ratio-based normalization method, instead of using individual ncRNAs as biomarkers, we calculated the ratio of any two ncRNAs in the same sample and used the resulting ratios as biomarkers.

In the first aspect, the present invention provides a method for identifying a diagnostic biomarker, comprising steps of:

(1) Determining species of ncRNAs in a biological sample;
(2) Determining an amount of each ncRNAs in the biological sample;
(3) Calculating a ratio of any two ncRNAs in each biological sample;
(4) Calculating a ratio of any two ncRNAs group average based on the average value of each ncRNA in multiple biological samples group;
(5) Identifying optimal ncRNAs pair by using support vector machine recursive feature elimination (SVM-RFE) algorithm; and
(6) Using the ratio of ncRNA pair as a standard to classify the biological sample.

In one embodiment, said biological sample is plasma, said biological sample group at least includes normal sample group, disease sample group, preferably said disease group includes cancer sample group, benign tumor sample group, and said ncRNAs comprise miRNA, snoRNA, piRNA, siRNA and tRNA.

In another embodiment, step (1) comprises RNA extraction and small molecular RNA sequencing. RNA extraction includes but not limited to extracting from plasma with TRIzol reagent, adding SiO₂ film to block adsorption within column and then collecting the absorbed RNA after washing.

In another embodiment, said small molecular RNA sequencing includes but not limited to sequencing by SMARTer smRNA-seq method, specifically including 3′ adapter ligation, 5′ RT primer annealing, 5′ adapter ligation, reverse transcription (RT), and PCR amplification for RNA sample.

In another embodiment, step (2) is to determine the amount of plasma ncRNAs by RT and quantitative PCR (RT-qPCR), preferably using Taqman miRNA kit.

In another embodiment, step (3) is to evaluate the ratio of two small ncRNAs (ncRNA1/ncRNA2) in the same sample using comparative CT method (2^{-ΔCT}), in which ΔCT=CT ncRNA1−CT ncRNA2, based on RT-qPCR data.

In another embodiment, step (4) comprises log 2 transforming the ncRNA concentration in plasma, using unpaired T-Test in SPSS 20.0 software to compare mean ncRNA ratios among different biological sample groups, with the significant p-value level set at 0.05.
In another embodiment, in step (4) said biological group at least includes normal sample group, disease sample group, preferably said disease group includes cancer sample group, e.g., lung adenocarcinoma sample group, or benign tumor sample group.

In another embodiment, in step (5), support vector machine recursive feature elimination (SVM-RFE) algorithm includes:
a. Initializing the dataset to contain features,
b. Training an SVM on the dataset,
c. Ranking features according to cᵢ=(wᵢ)²,
d. Eliminating the lower-ranked 50% of the features,
e. Returning to step b.

In another embodiment, the identified markers for diagnosis are used for classification of clinical samples to be measured to judge whether an individual from which the clinical sample is derived is suffering from said disease.

In another embodiment, said ncRNA may be replaced with another biomarker, including mRNA, DNA, protein and metabolite.

In the second aspect, the present invention provides a biomarker pair for diagnosis identified and obtained by the method of the present invention.

In one embodiment, the biomarker pair is selected from a group consisting of miR378a-3p/miR126-5p, sno-DR119/tRNA-Thr-ACG, sno-ACA33/miR378a-3p, tRNA-Thr-ACG/sno-U57, and tRNA-Thr-ACG/miR378a-3p.

In the third aspect, the present invention provides a method for diagnosis of lung adenocarcinoma, comprising:

(1) Detecting quantitatively ncRNAs pair associated with lung adenocarcinoma in a sample to be measured, calculating a ratio of ncRNAs pair, wherein said ncRNAs pair associated with lung adenocarcinoma is one identified and obtained by the method of the present invention;

(2) Comparing the ratio of ncRNAs pair with the ratio of ncRNAs pair group average in lung adenocarcinoma sample group, and the ratio of ncRNAs pair group average in normal sample group;

(3) Classifying the samples to be measured into lung adenocarcinoma sample group and normal sample group, and then diagnosing or auxiliary diagnosing whether individuals from which said biological samples are derived are suffering from lung adenocarcinoma.

In one embodiment, the method for diagnosis of lung adenocarcinoma further comprises after step (3):

(4) Based on the clinically confirmed results of said samples to be measured, the ratio of ncRNAs pair in the sample is used to calibrate the ratio of ncRNAs pair group average in lung adenocarcinoma sample group or the ratio of ncRNAs pair group average in normal sample group.

In another embodiment, the method for diagnosis of lung adenocarcinoma further comprises after step (4):

(5) Using the calibrated ratio of ncRNAs pair group average in lung adenocarcinoma sample group and in normal sample group to diagnose next lung adenocarcinoma sample.

In another embodiment, in the method for diagnosis of lung adenocarcinoma, said ncRNAs pair associated with lung adenocarcinoma is selected from a group consisting of miR378a-3p/miR126-5p, sno-DR119/tRNA-Thr-ACG, sno-ACA33/miR378a-3p, tRNA-Thr-ACG/sno-U57 and tRNA-Thr-ACG/miR378a-3p.

The identified biomarkers of the present invention may be used to classify said biological samples in terms of health conditions of individuals from which the samples are derived, and have a massive value in the clinical application according to the principle of the present invention. The method for identifying a biomarker of the present invention is not only used in diagnosis of diseases but also in a general detection for the non-diagnostic purpose.

Relative to the prior art, the present invention achieves the positive effects as follows:

(1) The present invention provides a ratio based method for normalization circulating ncRNA data by using ratio of ncRNAs as classification criteria. Relative to a single ncRNA, ratio of ncRNAs may have more choices, more significant difference and more accurate reflection of the true vales.

We first calculate the ratio of any two ncRNAs in the same sample, then compare the ratio expression levels between different groups rather than compare the level of a single ncRNA. Since the two ncRNAs are simultaneously measured in the same sample under the same condition such as collection, storage and isolation, and PCR or sequencing processing, the relative expression level in ratio of the two ncRNAs will reflect the true value for comparison.

(2) It is mathematically proven that the ratio based normalization method of the present invention is logically correct, which is independent of any external or internal reference control molecules and superior to any existing external or internal control based normalization methods. This ratio strategy provides a practical method in terms of clinical application of circulating ncRNAs as biomarkers of human diseases. We were also first to mathematically prove that the ratio based normalization method is better than any methods based on internal or external control normalization factors. The internal or external control normalization based method has two assumptions. First, it assumes that the measured miRNA and the internal control in the same sample are influenced by the same systematic factors; second, it assumes that the true internal control values across different samples are the same. The ratio based method only assumes different miRNAs in the same sample share the same systematic factors, therefore, clearly we mathematically prove that the ratio based method is better than reference control based normalization method because it is hard to know whether the second assumption is true.

(3) The ratio based biomarker primer pair has increased chances of finding clinically meaningful biomarkers. A ratio based normalization method can find more significantly differentially ncRNA candidate markers between disease groups. It is also logically easy to understand, for example, giving the ratio of miRNA1/miRNA2 in the healthy normal and cancer groups, if miRNA1 has an upregulated fold change in cancer vs normal, and miRNA2 has a downregulated fold change in cancer vs normal. The fold change of miRNA1/miRNA2 between cancer and normal should be bigger than that of miRNA1 or miRNA2 alone. So ratio based method will increase our chance to find clinically useful biomarkers when sometimes we may not be able to find significantly changed single markers.

(4) Initially we have found that a panel of circulating 5 paired ncRNA ratios could separate lung adenocarcinoma from normal healthy control with 100% prediction accuracy. We not only tested miRNAs and also measured other types of ncRNAs such as snoRNAs and tRNAs.

(5) The method for diagnosis of lung adenocarcinoma of the present invention may continuously validate the ratio of mean values of ncRNAs pair in lung adenocarcinoma sample group and/or the ratio of mean values of ncRNAs pair in normal sample group with growing data of new cases as they increase clinically, so as to increase the accuracy and reliability of the diagnostic method.

### BRIEF DESCRIPTION OF THE DRAWINGS

FIG. 1: Read number of spiked-in external control Cel-miR-54

There are a total of 7 pooling plasma samples that were used for small-RNA sequencing. Equal amount of synthesized C. elegans external of Cel-miR-54 was added into the pooling samples before RNA isolating and sequencing. Each pool contained 15 mixed samples. LC represents normal healthy control (2 pooling samples), BE represents Benign (2 pooling samples), AD represents lung adenocarcinoma (2 pooling samples) and SC represents squamous cell lung cancer (1 pooling sample).

FIGS. 2A, 2B, and 2C: RT-qPCR CT values of external and internal reference controls in cancer and non-cancer samples

CT values were sorted based on the number of a total of 129 samples including lung cancer, benign and normal healthy control plasma samples. (FIG. 2A) CT values of external C. elegans Cel-miR-54 across 129 samples. (FIG. 2B) CT values of endogenous reference control has-miR-191 across 129 samples. (FIG. 2C) CT values of endogenous reference control of averaged has-Let-miR-let 7d/g/i across 129 samples.

FIG. 3: Differentiated single ncRNA numbers and ncRNA ratio numbers

X-axis represents the total measurable features (either miRNA or miRNA ratios), the differentiated number of normal healthy control vs lung adenocarcinoma, normal healthy control vs benign and benign vs lung adenocarcinoma. An unpaired t-test was used to identify differentiated miRNA or miRNA ratios. P value<−0.05 and fold change cut-off was 2.0. Ratio was calculated between any two miRNAs in the same sample.

FIGS. 4A, 4B, 4C, and 4D: Expression value of representative ncRNA ratios in the adenocarcinoma lung cancer and normal samples

Each individual ncRNA in the plasma was measured using quantitative real time RT-PCR, the ratio of two ncRNAs in the same sample was calculated as (2^{-ΔCT}), in which ΔCT=CT ncRNA1−CT ncRNA2. So −ΔCT=log₂ (ncRNA1/ncRNA2). (FIG. 4A) miR378a-3p/miR126-5p. (FIG. 4B) sno-DR119/tRNA-Thr-ACG. (FIG. 4C) tRNA-Thr-ACG/sno-U57. (FIG. 4D) tRNA-Thr-ACG/miR378a-3p (***p<0.001).

FIG. 5: Separation of plasma samples of adenocarcinoma lung cancer from normal control group by 5 paired ncRNA ratio markers

Two ways hierarchical clustering based on these 5 paired markers was performed to show the group clustering. 50 lung adenocarcinoma samples (Adeno) and 29 normal healthy control samples (Normal) were used for real time RT-qPCR. Color bar shows the expression value of the markers.

### EXAMPLES

The present invention is further illustrated by the following examples, which are merely to describe rather than restrict the scope of the present invention. The experimental conditions usually follow the conventional ones or as suggested by the manufactures, and thus are not specially noted in the following examples. All the technical terms in the Description have the same meanings as known by those skilled in the art, unless defined otherwise. Further, any methods or materials similar to those recorded in the Description may also apply to the method of the present invention. The preferable methods and materials in the present invention are only exemplified.

Specific technologies or conditions not noted in the examples may follow those as described in the documents in the prior art, or as suggested in the product instructions. All the reagents or instruments not noted in the Description may be commercially purchased as conventional means.

### Example 1: Patient Cohorts and Plasma Samples Collection

We enrolled approximately 1,250 patients in our Lung Cancer Biorepository at Beijing People's Hospital from 2004 to 2010 and from these we selected a sub-cohort of 130 patients, including 50 with early staged (stage I, II) lung adenocarcinoma, and 15 SCC, and benign cases, and 30 normal individuals for this pilot study. The early stage adenocarcinoma and SCC patient inclusion criteria included the disease confined to the chest without evidence of distant metastases; no preoperative chemo- or radiotherapy within 1 year of our initial blood sampling; and a minimum of 2 years of clinical follow-up data. Patients with benign lesions include participants with a range of non-neoplastic pulmonary disorders (e.g. granulomas, hamartomas, and inflammatory lesions) as indicated in low-dose computed tomography (LDCT) screen. All benign participants and normal individuals were followed with annual LDCT and remained cancer-free for a minimum 2-year follow-up. Demographic information for these patients and controls is listed in Table 1.

TABLE 1
Patients' characteristics of all samples used
in both training and validation stage

| | Adeno. | SCC | Benign | Normal |
|---|---|---|---|---|
| | n = 50 | n = 15 | n = 35 | n = 29 |
| Age*, yr | | | | |
| mean | 66.32 | 64.04 | 62.06 | 60.59 |
| SD | 7.85 | 8.01 | 9.15 | 8.09 |
| range | 49-80 | 48-82 | 42-77 | 50-76 |
| Gender, n | | | | |
| (%) | | | | |
| male | 21 (42.0) | 8 (53.5) | 18 (51.4) | 13 (44.8) |
| female | 29 (58.0) | 7 (46.5) | 17 (48.6) | 16 (55.2) |
| Race | | | | |
| Caucasian | 50 | 15 | 35 | 29 |
| Non | 0 | 0 | 0 | 0 |
| Caucasian | | | | |
| Tumor | | | | |
| Stage n (%) | | | | |
| Stage 0-1 | 28 (56.0) | 10 (66.6) | | |
| Stage 2 | 22 (44.0) | 5 (33.4) | | |

Cancer, benign and normal samples were approximately age-, race-, gender- and smoking status-matched as much as possible. The cohort of normal subjects was also described as a “high-risk” population, in which all the healthy subjects have had a smoking history of more than 30 pack-years and quit less than 15 years before randomization. All patient data were acquired with written formal consent and in absolute compliance with the institutional review board at Beijing People's Hospital.

All plasma samples were collected using EDTA-anticoagulative tubes and centrifuged for at 4000 RPM for 10 min, followed by a 15 min high-speed centrifugation at 12,000 RPM to completely remove cell debris. The supernatant plasma was stored at −80° C. until analysis. All samples were collected when the diagnosis was firstly made.

### Example 2: RNA Isolation and Sequencing

RNA isolation was described previously. Total RNA, including small RNAs from plasma, was isolated by using the miRNeasy kit (Qiagen, Valenciz, Calif.) with minor modifications. In brief, 0.5 ml plasma should be diluted 1:1 with RNase-free water (totally 1 ml) to get fully phase separation. 3 mL of TRIzol® LS Reagent was added to per 1 mL of sample volume. The sample was mixed in a tube, vortex 10s, incubate at room temperature for 15 mins (totally 4 ml) to permit complete dissociation of the nucleoprotein complex. Centrifuge homogenized solution at 12,000×g for 10 minutes at 4° C. Transfer the cleared supernatant (containing RNA) to a new tube. Add 0.8 mL of chloroform into the transferred supernatant. After mixing vigorously for 15 seconds, the sample was then centrifuged at 12,000 g for 15 min. The upper aqueous phase was carefully transferred to a new collection tube, and 2.5 vol of ethanol was added. The sample was then applied directly to a silica membrane adsorption column and the RNA was bound and cleaned by using buffers provided by the manufacturer to remove impurities. The immobilized RNA was then collected from the membrane with 16 μl RNase-free water (pre-warm up at 80° C.).

In this study we used an Illumina next generation sequencing to sequence plasma samples at City of Hope in California. Briefly, to save cost and samples, we first conducted small RNA sequencing (smRNA-seq) to identify plasma microRNAs and some other circulating small non-coding RNAs (sncRNAs) in 7 pooling samples including 30 high-risk healthy individuals (Normal), 30 individuals with benign nodule lesions (Benign), 30 lung adenocarcinoma, and 15 SCC. Normal, benign, and cancer samples are all age, sex, race and smoking status matched. The samples were prospectively collected from the training cohort (from Beijing People's Hospital, but unfortunately, we lost one normal sample when we handled PCR). Two pooling samples (15 samples per pool) for each group were used for smRNA-seq except SCC, at about 500 μl of equally mixed plasma in each pooling sample. About 20 million reads per sample with about 90% of reads aligned to human genome was produced.

For the library preparation, 6 μl of the eluates from the serum RNA isolation was used. Preparation was performed following the Illumina protocol with minor modifications. A miRNA library is made from each RNA sample by 3′ adapter ligation, 5′ RT primer annealing, 5′ adapter ligation, reverse transcription, and PCR amplification. Libraries were then pooled in batches of 12 samples in equal amounts and clustered with a concentration of 10.5 pmol in one lane each of a single read flowcell using the cBot (Illumina). Sequencing of 50 cycles was performed on a HiSeq 2500 (Illumina). Demultiplexing of the raw sequencing data and generation of the FASTQ files were done using CASAVA v. 1.8.2.

From the FASTQ files the 3′ sequencing adapter will be removed by a local alignment of the adapter to the sequenced reads. We used the cut adapt software to remove the 3′ adaptor. All sequences having a length less than 15 bps after adapter removal were discarded. The reads in each library were summarized to tags in a quantified FASTA format. The FASTA reads were then mapped to the genome under consideration with bowtie. To eliminate the ambiguous mapping hits, only the uniquely mapped loci with the fewest alignment mismatches were reported allowing a maximum of two mismatches. Expression profiles in different libraries were determined by mapping the clean reads back to human ncRNAs. For each mapping locus annotations are derived from several ncRNA databases.

### Example 3: RT and Real Time PCR

NcRNAs were measured using Taqman miRNA assay kits (Applied Biosystems, USA) according to the manufacturer's protocol. Briefly, about 30 ng enriched RNA was reverse transcribed with a TaqMan ncRNA Reverse Transcription Kit (Applied Biosystems, USA) in a 15 μL reaction volume. Expression levels of ncRNAs were quantified in triplicate by qRT-PCR using human TaqMan MicroRNA Assay Kits (Applied Biosystems, USA) on Eppendorfiplex 4 system (Eppendorf North America, Hauppauge, N.Y.). To bypass the normalization issue, we use the same ratio strategy instead of normalizing to reduce the experimental variations.

### Example 4: Statistical Analysis

The ratio was calculated of any two ncRNAs in the same sample for both the sequencing data and RT-qPCR data. For RT-qPCR data, if a CT value is bigger than 40, it was changed to 40. Then expression levels of ratio of two small ncRNA (ncRNA1/ncRNA2) were evaluated using comparative CT method (2^{-ΔCT}), in which ΔCT=CT ncRNA1−CT ncRNA2 in the same sample. We used the unpaired T-Test in SPSS 20.0 software to compare mean ncRNA ratios between adeno. case, benign patient, and normal control groups after the ncRNA concentrations of plasma were log 2 transformed, with the significant p-value level set at 0.05. Chi-Square test in SPSS 20.0 software was used to compare the distribution of training and validation stages with regards to gender, race and tumor stage and t-test to age. The significant p-value level was set at 0.05 for all results. Support vector machine recursive feature elimination (SVM-RFE) algorithm was used to select best ncRNAs. SVM-RFE is an algorithm for selecting a subset of features for a particular learning task. The basic algorithm is the following: (1) initialize the dataset to contain features, (2) train an SVM on the dataset, (3) Rank features according to cᵢ=(wᵢ)², (4) eliminate the lower-ranked 50% of the features, (5) return to step (2). At each RFE step (4), a number of features are discarded from the active variables of an SVM classification model. The features are eliminated according to a criterion related to their support for the discrimination function, and the SVM is re-trained at each step. Selected ncRNA ratios from the feature selection algorithm were used for classification using support vector machines (SVMs). A 5-fold cross-validation procedure was for both internal and external validations. We used the prediction performance metrics including sensitivity, specificity, positive predictive value (PPV), negative predictive value (NPV) and area under ROC curve (AUC) to judge the performance of the prediction accuracy.

### Example 5: A Ratio Based Normalization Method for Circulating ncRNA Profiling Data is Independent of any Internal or External Normalization Controls

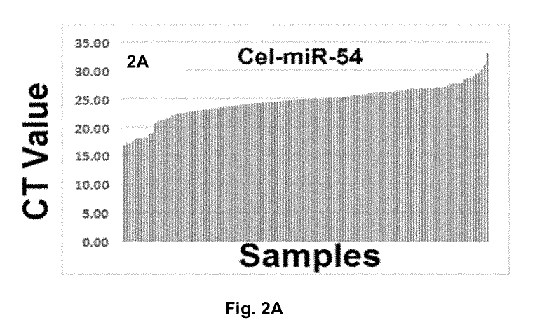

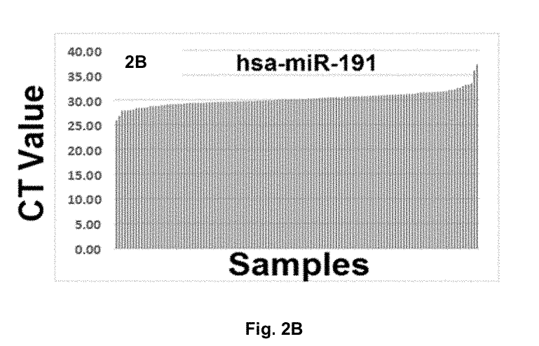

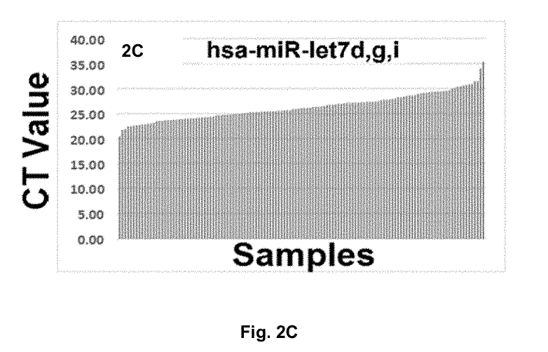

Since that neither of the external and internal controls (FIG. 2) were reliable for normalizing circulating ncRNA profiling data, we next tested a ratio-based normalization method for normalizing circulating ncRNA profiling data. We first calculate the ratio of any two ncRNAs in the same sample, then compare the ratio of expression levels between different groups rather than compare the level of a single ncRNA. Taking miRNA and internal control (IC) as examples (see Table 2).

TABLE 2
A ratio based normalization method

| | | | | Fold |
|---|---|---|---|---|
| Row | MiRNAs | Normal | Cancer | change* |
| 1 | miRNA1 | 4 | 8 | 2 |
| 2 | miRNA2 | 8 | 4 | −2 |
| 3 | Internal Control 1 (IC1) | 2 | 4 | 2 |
| 4 | Internal Control 2 (IC2) | 4 | 2 | −2 |
| 5 | miRNA1/IC1 | 4/2 = 2 | 8/4 = 2 | 1 |
| 6 | miRNA2/IC1 | 8/2 = 4 | 4/4 = 1 | −4 |
| 7 | miRNA1/IC2 | 4/4 = 1 | 8/2 = 4 | 4 |
| 8 | miRNA2/IC2 | 8/4 = 2 | 4/2 = 2 | 1 |
| 9 | (miRNA1/IC1)/(miRNA2/ | 2/4 = 0.5 | 2/1 = 2 | 4 |
| | IC1) = miRNA1/miRNA2 | | | |
| 10 | (miRNA1/IC2)/(miRNA2/ | 1/2 = 0.5 | 4/2 = 2 | 4 |
| | IC2) = miRNA1/miRNA2 | | | |
| 11 | miRNA1/miRNA2 | 4/8 = 0.5 | 8/4 = 2 | 4 |

*Positive value means upregulation in cancer, and negative value means downregulation in cancer.

The expression value of miRNA1 in normal and cancer samples is 4 or 8, respectively, the fold change between normal and cancer is 2 (row 1). The expression value for internal control 1 (IC1) in normal and cancer samples is 2, or 4 respectively (row 3). If miRNA1 is normalized by IC1, the fold change between normal and cancer is 1 (row 5); if miRNA1 is normalized by internal control 2 (IC2), the fold change between normal and cancer is 4 (means upregulation 4 times, row 7). Thus, even without normalization (row 1) or using different internal controls (IC1 or IC2), we observe different fold changes between normal and cancer samples. Similar to miRNA1, we also observed different fold changes results of miRNA2 (see rows 2, 6 and 8). If we first normalize miRNA1 and miRNA2 by IC1, then calculate the ratio between IC1 normalized miRNA1 and miRNA2 values, the value of the normal sample is 0.5 and the cancer sample value is 2, and the fold change is 4 (row 9). Interestingly, if we normalize miRNAs by IC2 (row 10) or without any normalization (row 11), then calculate the ratio of the two miRNAs in the same samples, the ratio value of normal sample is still 0.5 (rows 10 and 11), and the value of cancer sample is 2 too (rows 10 and 11), the fold change is still 4 (rows 10 and 11). The results indicate no matter what kind of internal controls we use, the ratio of any two miRNAs in the same sample will not change. So we can just calculate the ratio of any two miRNAs in the same sample for normalization of miRNA profiling data (row 11), which is independent of any internal or external controls.

### Example 6: A Ratio Based Normalization Method is Mathematically Correct

From table 2, we already know that a ratio based normalization is efficacious. Here we would like to mathematically show the method is also logically correct. Again, we use miRNA as an example. Our ultimate goal is to get the biologically true miRNA value (truemiRNA), however, usually our observed miRNA (OBSmiRNA) value achieved from an experiment is not the true value. Actually the OBSmiRNA value is the result of truemiRNA impacted by different systematic factor. In the case of RT-qPCR, the systematic factors could include RNA isolation (I), reverse transcription (R), PCR (P), different time (T) and so on. Therefore in a specific sample such as S1, we could set

OBSmiRNA1=TruemiRNA*Is1*Rs1*Ps1*Ts1 (1)

Similarly, we assume the systematic factors in the same sample for the miRNA2 is the same, the OBSmiRNA2 in the same S1 could also set as

OBSmiRNA2=TruemiRNA2*Is1*Rs1*Ps1*Ts1 (2)

So,

OBSmiRNA1/OBSmiRNA2=TruemiRNA1/TruemiRNA2 (3)

From row 3, we can clearly see that the ratio of observed two miRNAs in the same sample will equal to the true ratio value of the two true miRNAs. Thus, we mathematically prove that the ratio value of two observed miRNAs in the same sample can reflect the true biological value of the two miRNAs that we want to measure.

The PCR value is CT value, which actually is a log value. From the formula (4), we can see that the log ratio value of two miRNAs in factor is the minus of two CT values of the two miRNAs, which will make the calculation even easier and more convenient for clinically practice use based on RT-qPCR data.

Log₂(OBSmiRNA1/OBSmiRNA2)=Log₂(2^{-CTmiRNA1}/2^{-CTmiRNA2})=Log₂(2^{-CTmiRNA1}/2^{-CTmiRNA2})=Log₂(2^{-CTmiRNA1+CTmRNA2})CTmiRNA2−CTmiRNA1 (4)

### Example 7: Mathematically the Ratio Based Normalization Method is Better than Internal or External Control Normalization Method

Even though we have mathematically proved that the ratio based normalization method is logically correct, people may also question to our assumption that the systematic factors are the same for different miRNAs in the same sample. In theory it is right because those two miRNAs are in the same sample and should be impacted by the same systematic factors. Actually the reference control based normalization methods do the same.

Mathematically further analyzing and comparing the ratio based normalization method with internal or external control normalization method:

OBSmiRNA1S1=TruemiRNA1S1*Is1*Rs1*Ps1*Ts1 (1)

We can set

Is1*Rs1*Ps1*Ts=Factor1 (2)

Then, the true value of miRNA1 in sample 1 (S1)

TruemiRNA1S1=OBSmiRNA1S1/Factor1 (3)

Similarly for the true value of miRNA1 in sample2 (S2)

TruemiRNA1S2=OBSmiRNA1S2/Factor2 (4)

Similarly for true value of internal control (IC) in sample 1 (S1) and sample 2 (S2)

TrueICS1=OBSICS1/Factor1 (5)

TrueICS2=OBSICS2/Factor2 (6)

So, based on (5) and (6), we can get

Factor1=OBSICS1/TrueICS1 (7)

Factor2=OBSICS2/TrueICS2 (8)

Let's replace Factor 1 (7) to (3) and Factor 2 (8) to (4), we should get

TruemiRNA1S1=(OBSmiRNA1S1/OBSICS1)*TrueICS1 (9)

TruemiRNA1S2=(OBSmiRNA1S2/OBSICS2)*TrueICS2 (10)

Suppose

TrueICS1=TrueICS2 (11)

We can consider

TruemiRNA1S1=OBSmiRNA1S1/OBSICS1 (12)

TruemiRNA1S2=OBSmiRNA1S2/OBSICS2 (13)

The formulae of (12) and (13) are the currently external or internal control based normalization method. It considers that normalized value of an overserved miRNA by the internal control (IC) in the same sample is the true value of the miRNA. To achieve the value, it has two assumptions. First, it assumes that the measured miRNA and the internal control in the same sample are influenced by the same systematic factors (see (2) and (5) or (4) and (6)); second, it assumes that the true internal control values across different samples are the same (see (11)). However, it is hard to know whether the second assumption is true or not. The ratio based method only assumes different miRNAs in the same sample share the same systematic factors, therefore, clearly we mathematically prove that the ratio based method is better than reference control based normalization methods.

### Example 8: Ratio Based Normalization Method can Find More Significantly Differentially ncRNA Candidate Markers Between Disease Groups

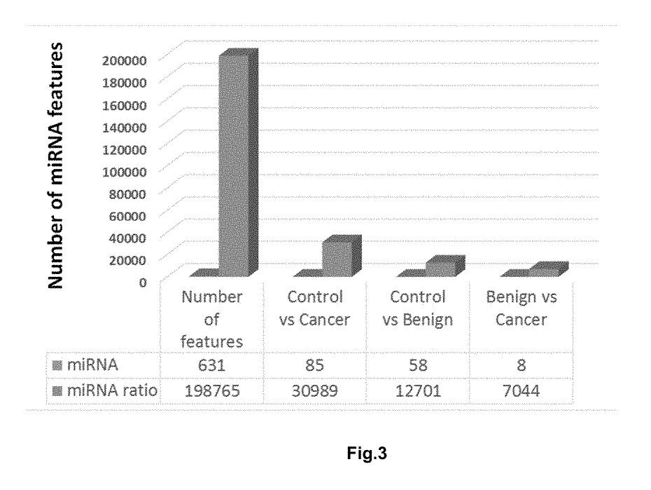

Originally we proposed the ratio based normalization method on circulating RT-qPCR data. Yet, the external spiked-in control failed to work for normalizing sequencing data. For example, given miRNA with at least 20 reads for an miRNA, we found 631 mature miRNAs in the sequenced samples. Next, we calculated the ratio of any two miRNAs in a sample, we could surprisingly get 198,765 ratios (FIG. 3), which will substantially increase our candidate miRNAs to find different expressed paired ratio markers between disease groups. To provide a list of differentially expressed miRNA ratios, we further did differential expression analysis with comparison between cancer vs control, cancer vs benign, and benign vs control of the pooling samples. Based on fold change ≥2 and p value ≤0.05, we found a large number of significantly altered mature miRNA ratios (miRNA/miRNA) including 30,989 ratios between normal and cancer, 12,701 ratios between normal and benign, and 7,044 ratios between benign and cancer. These significantly changed ratio numbers are much more divergent than the measurements of single miRNAs between the 3 groups based on global median normalization for single miRNA data (FIG. 3).

### Example 9: Ratio Based ncRNA Biomarkers for Separating Healthy Control from Luna Adenocarcinoma

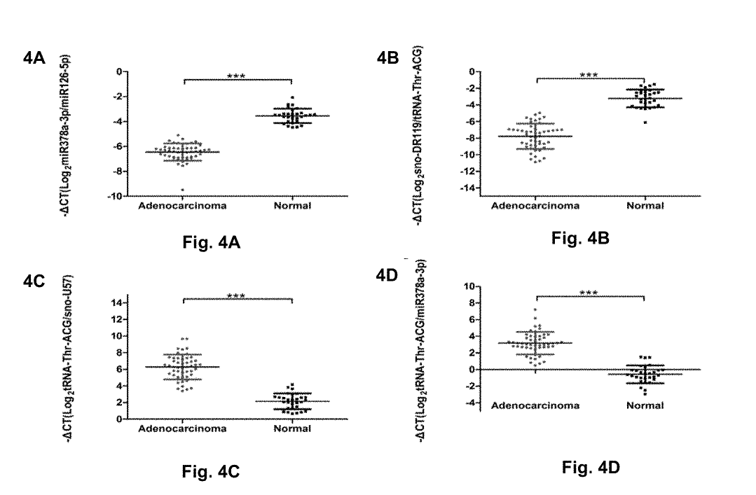

To test how these ratio based candidate ncRNAs distinguished lung cancer from non-cancer samples, initially we chose about 20 paired significantly ncRNA ratios in the comparison of control vs. cancer from sequencing data in 29 control, and 50 lung cancer adenocarcinoma samples at early stages with age, race, sex, and smoking status matched. Using support vector machine recursive feature elimination (SVM-RFE) feature selection and SVM classification algorithm, we found that with a combination of 5 ncRNA ratios, we could reach prediction accuracy of 100% for all measured parameters including sensitivity, specificity, positive predictive value (PPV), negative predictive value (NPV) and area under ROC curve (AUC). FIG. 4 shows the expression value of the representative ncRNA ratio markers in the 50 adenocarcinoma lung cancer and 29 normal samples.

### Example 10: Using Combined Ratios of Circulating ncRNA Pairs to Predict the Accuracy of Separating Luna Adenocarcinoma Sample from Normal Sample

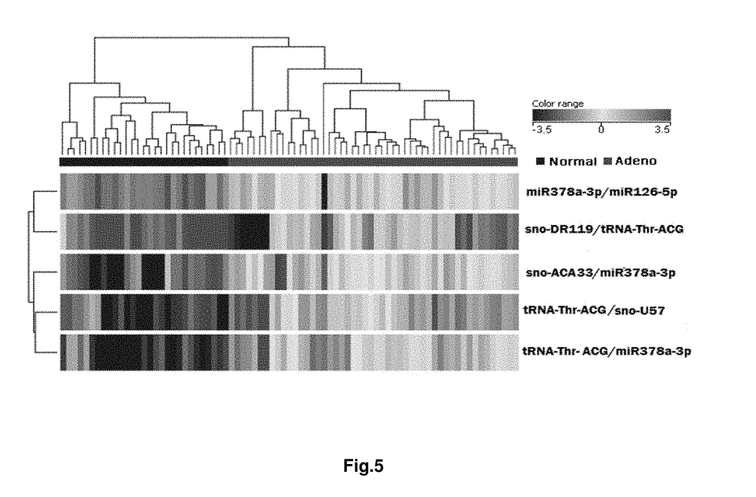

ncRNA in plasma of each individual is tested by real time RT-qPCR. Two ratios of ncRNAs in the same sample are calculated as 2-ΔCT, wherein ΔCT=CT ncRNA1−CT ncRNA2, and so, −ΔCT=log 2(ncRNA1/ncRNA2). After analysis, it is found that miR378a-3p/miR126-5p, sno-DR119/tRNA-Thr-ACG, tRNA-Thr-ACG/sno-U57, and tRNA-Thr-ACG/miR378a-3p ncRNA pairs may be used to remarkably and specifically separate lung adenocarcinoma and normal control (FIG. 5). FIG. 5 depicts that even with an unsupervised hierarchical clustering, the adenocarcinoma group could be separated from the normal control group without misclassification of a single sample.

Results of Examples 9 and 10 indicate that even with an unsupervised hierarchical clustering, the adenocarcinoma sample could be separated from the normal sample without misclassification of a single sample.

### Comparative Example 1: External C. elegans Cel-miR-54 was not a Good Control for Normalizing Circulating Small Molecular RNA Sequencing

In order to identify circulating small molecular ncRNA markers for detection of lung cancer, we performed whole genome level small ncRNA (smRNA-seq) using pooling samples based on human plasma samples to save cost and samples. We first conducted small RNA sequencing (smRNA-seq) to identify plasma microRNAs and some other circulating small non-coding RNAs (sncRNAs) in 7 pooling samples including 30 high-risk healthy individuals (healthy control), 30 individuals with benign nodule lesions, 30 early stage lung adenocarcinoma, and 15 squamous cell lung cancer (SCC). Each pool contained individual samples. Control, benign, and cancer samples are all age, sex, race, and smoking status matched. The samples were prospectively collected from Rush University Medical Center. Two pooling samples for each control, with benign and adenocarcinoma lung cancer and one pool for SCC. About 500 μl of equally mixed plasma in each pooling sample, were used for smRNA-seq. This was done using the Illumina next generation sequencing platform at City of Hope in California. About 20 million reads per sample were generated with about 90% of reads aligned to human genome.

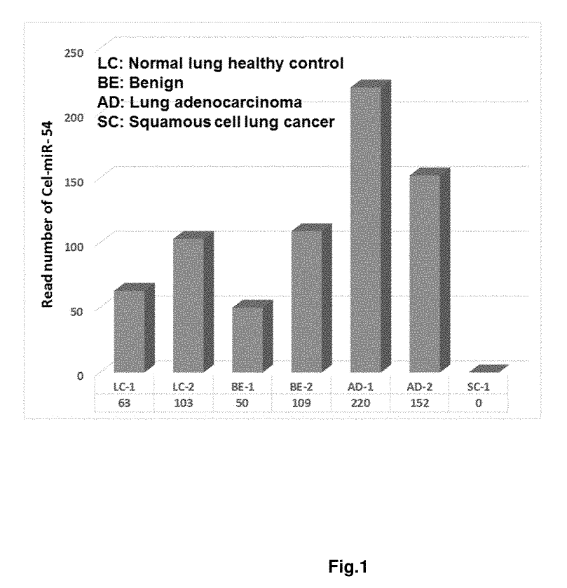

Since C-elegans Cel-miR-54 is not contained in the human body, it was used as an external control for the sequencing. Equal amount of Cel-miR54 was added into the pooling samples before RNA extraction. So we expected that we should get equal read number of cel-miR-54 in all the pooling samples. As shown in FIG. 1, the read number for cel-miR-54 was quite different across the 7 pooling samples. The highest number was 200 for one adenocarcinoma lung cancer pooling sample. However, we saw 0 reads from the SCC pooling sample. These data suggest that the external control Cel-miR-54 is not a reliable control for normalizing smRNA-seq data.

### Comparative Example 2: External C. elegans Cel-miR-54 was not a Good Control for Normalizing Circulating Quantitative RT-PCR (RT-qPCR) Small ncRNA

Next we tested if external C. elegans Cel-miR-54 is a good control for normalizing circulating quantitative RT-PCR (RT-qPCR) small ncRNA data. We selected 129 samples (29 healthy control, 50 adenocarcinoma lung cancer, 35 benign, and 15 SCC) to perform RT-qPCR of Cel-miR-54. Equal amount of Cel-miR-54 was added into the equal amount of plasma (200 μl) before RNA was isolated in the individual samples. As illustrated in FIG. 2A we found that the CT values of published external control Cel-miR-54 were quite unstable; the CT values ranged from about 14 to about 34. The highest and lowest CT values had 20 CT values difference, equal to around 40-fold difference from original data. Because the same amount of Cel-miR-54 was added, we expected to have approximately equal CT values across the samples. These additional experiments again support the conclusion that external Cel-miR-54 is not a consistent control for normalizing circulating RT-qPCR of small ncRNA data either.

### Comparative Example 3: Endogenous Controls were not Good for Normalizing Circulating Quantitative RT-PCR (RT-qPCR) Small ncRNA Data

Since we failed to use external control such as Cel-miR-54 to normalize circulating ncRNA RT-qPCR data, we sought whether we could use endogenous controls to normalize circulating ncRNA RT-qPCR data. Based on published reports, we chose has-miR-191 and has-miRNAs, Let-7d, Let-7g, and Let-7i as our endogenous controls. Based on the same amount of volume of RNA (about 2 μl) isolated from the same amount volume (200 μl) of plasma samples which were the same as we used for external control cel-miR-54 (FIG. 2A), we conducted RT-qPCR for the endogenous controls in the same 129 samples. As shown in FIG. 2, the CT values of published internal controls including has-miR-191 (FIG. 2B) and averaged has-MiRNAs, Let-7d, Let-7g and Let-7i (FIG. 2C) were also ranged quite differently and unstably expressed. Thus we think they are not suitable as reference controls for normalizing circulating ncRNA RT-qPCR data.

The above description intends not to restrict the scope of the present invention, and the present invention is not limited to such examples as well. Those skilled in the art may make some changes, modifications, substitutions or additions without departing from the spirit, which also fall into the scope of the present invention based on the claims appended here.

What is claimed is:

1. A method for identifying a diagnostic biomarker in a biological sample from a patient group, comprising the steps of:
(1) determining species of ncRNA in the biological sample;
(2) determining amount of each ncRNA species in the biological sample;
(3) calculating ratio of the amount of any two ncRNA species in the biological sample;
(4) calculating ratio of any two ncRNA species based on average of each ncRNA species from the patient group;
(5) identifying optimal ncRNA pairs using a support vector machine recursive feature elimination (SVM-RFE) algorithm; and
(6) using the ratio of ncRNA pair as a standard to classify the biological sample.

2. The method of claim 1, wherein said ncRNA is miRNA, snoRNA, piRNA, siRNA or tRNA.

3. The method of claim 1, wherein in step (1) the ncRNA species is determined by RNA extraction and small molecular RNA sequencing.

4. The method of claim 1, wherein in step (2) the amount of plasma ncRNAs is determined by quantitative detection of RT-qPCR.

5. The method of claim 1, wherein in step (3) the ratio of two ncRNAs (ncRNA1/ncRNA2) in the same sample is determined using a comparative CT method (2^{-ΔCT}), in which ΔCT=CT ncRNA1−CT ncRNA2, based on RT-qPCR data.

6. The method of claim 1, wherein in step (4) the ration determination comprises log 2 transforming the plasma ncRNA concentration, using unpaired T-Test in SPSS 20.0 software to compare group average ncRNA ratios among different biological sample groups, with the significant p-value level set at 0.05.

7. The method of claim 1, wherein in step (4) said biological groups include at least a normal sample group and a disease sample group.

8. The method of claim 1, wherein in step (5), the support vector machine recursive feature elimination (SVM-RFE) algorithm includes the steps of:
a. initializing the dataset to contain features,
b. training an SVM on the dataset,
c. ranking the features according to cᵢ=(wᵢ)²,
d. eliminating the lower-ranked 50% of the features; and
e. returning to step b.

9. The method of claim 1, wherein the identified biomarkers for diagnosis are used for classification of clinical samples to be measured to judge whether an individual from which the clinical sample is derived is suffering from a disease.

10. A biomarker pair for diagnosis identified and obtained by the method of claim 1.

11. The biomarker pair for diagnosis of claim 10, selected from the group consisting of miR378a-3p/miR126-5p, sno-DR119/tRNA-Thr-ACG, sno-ACA33/miR378a-3p, tRNA-Thr-ACG/sno-U57, and tRNA-Thr-ACG/miR378a-3p.

12. A method for diagnosis of lung adenocarcinoma, comprising the steps of:
(1) detecting quantitatively ncRNAs pair associated with lung adenocarcinoma in a sample to be measured, calculating a ratio of ncRNAs pair, wherein said ncRNAs pair associated with lung adenocarcinoma is one identified and obtained by the method of claim 1;
(2) detecting the ratio of ncRNAs pair with the ratio of ncRNAs pair group average in lung adenocarcinoma sample group and the ratio of ncRNAs pair group average in normal sample group;
(3) classifying the samples to be measured into lung adenocarcinoma sample group or normal sample group, and then diagnosing or auxiliary diagnosing whether individuals from which said biological samples are derived are suffering from lung adenocarcinoma.

13. The method for diagnosis of lung adenocarcinoma of claim 12, further comprising after step (3) the following step (4):
(4) based on the clinically confirmed result of said sample to be measured, the ratio of ncRNA pairs in the sample is used to calibrate the ratio of a ncRNA pair group average in a lung adenocarcinoma sample group or the ratio of a ncRNAs pair group average in a normal sample group.

14. The method for diagnosis of lung adenocarcinoma of claim 13, further comprising after step (4) the following step (5):
(5) using the calibrated ratio of the ncRNA pair group average in lung adenocarcinoma sample group and in normal sample group to diagnose a further lung adenocarcinoma sample.

15. The method for diagnosis of lung adenocarcinoma of claim 12, wherein said ncRNAs pair associated with lung adenocarcinoma is selected from the group consisting of miR378a-3p/miR126-5p, sno-DR119/tRNA-Thr-ACG, sno-ACA33/miR378a-3p, tRNA-Thr-ACG/sno-U57 and tRNA-Thr-ACG/miR378a-3p.
